SEQUENCE LISTING (6 sequences)

SEQ ID NO: 1 - DNA, 24 bp, Mus musculus
ggcgcaaagcaccgaagaaatgtt

SEQ ID NO: 2 - DNA, 24 bp, Mus musculus
ccatggcatcgcgcttatccattt

SEQ ID NO: 3 - DNA, 20 bp, Mus musculus
ggcagcctgtgagacctttg

SEQ ID NO: 4 - DNA, 21 bp, Mus musculus
gcattggaagtgaagcgtttc

SEQ ID NO: 5 - DNA, 24 bp, Mus musculus
tcaacagcaactcccactcttcca

SEQ ID NO: 6 - DNA, 24 bp, Mus musculus
accctgttgctgtagccgtattca

(19) United States
(12) Patent Application Publication
(10) Pub. No.: US 2011/0206696 A1
(43) Pub. Date: Aug. 25, 2011

(54) METHODS FOR INHIBITING BONE LOSS AND BONE METASTASIS

(75) Inventors: William Frazier, St. Louis, MO (US); Õzge Uluckan, Madrid (ES); Katherine Weilbaecher, St. Louis, MO (US)

(73) Assignee: THE WASHINGTON UNIVERSITY, St. Louis, MO (US)

(21) Appl. No.: 13/013,512

(22) Filed: Jan. 25, 2011

Related U.S. Application Data
(60) Provisional application No. 61/297,877, filed on Jan. 25, 2010.

(51) Int. Cl. A61K 39/395; A61P 19/08; A61P 35/00

(52) U.S. Cl. 424/172.1

(57) ABSTRACT

The invention encompasses methods of inhibiting bone loss and bone metastasis.

### CROSS REFERENCE TO RELATED APPLICATIONS

This application claims the priority of U.S. provisional application No. 61/297,877, filed Jan. 25, 2010, which is hereby incorporated by reference in its entirety.

### GOVERNMENTAL RIGHTS

This invention was made with government support under HL54390 and R01-CA097250 awarded by the National Institutes of Health. The government has certain rights in the invention.

### FIELD OF THE INVENTION

The invention encompasses methods of inhibiting bone loss and bone metastasis.

### BACKGROUND OF THE INVENTION

Bone metastases cause hypercalcemia, bone loss, fractures, and pain and are thus a significant cause of morbidity and mortality in cancer patients. Several tumor cell types (e.g., breast and prostate carcinomas and melanomas) metastasize to bone and lead to bone degradation via activation of bone-resorbing osteoclasts. Osteoclasts are formed from the fusion of monocytes/macrophages and are characterized by their large size, the presence of multiple nuclei, and positive staining for tartrate-resistant acid phosphatase (TRAP). Two cytokines, macrophage colony-stimulating factor (M-CSF) and receptor activator of nuclear factor-KB ligand (RANKL), are necessary for the survival and differentiation of macrophages into osteoclasts in vitro and in vivo.

The presence of tumor cells in the bone microenvironment results in osteoclast and osteoblast recruitment and activation. This activation stimulates the release of growth factors from stromal cells as well as from the bone matrix, which further promote tumor growth in bone. This is known as the vicious cycle of bone metastasis. Hence, there is a need in the art for methods of inhibiting bone metastasis and bone loss.

### SUMMARY OF THE INVENTION

One aspect of the present invention encompasses a method of inhibiting bone loss, the method comprising blocking CD47 signaling.

Another aspect of the present invention a method of inhibiting bone metastasis, the method comprising blocking CD47 signaling.

Yet another method of inhibiting osteoclast differentiation, the method comprising blocking CD47 signaling.

Other aspects and iterations of the invention are described more thoroughly below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

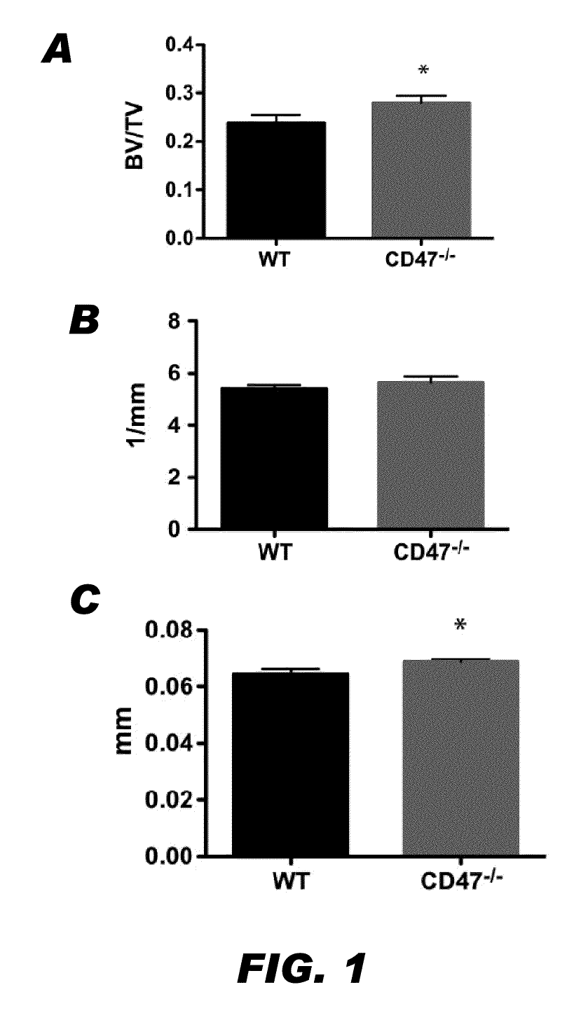

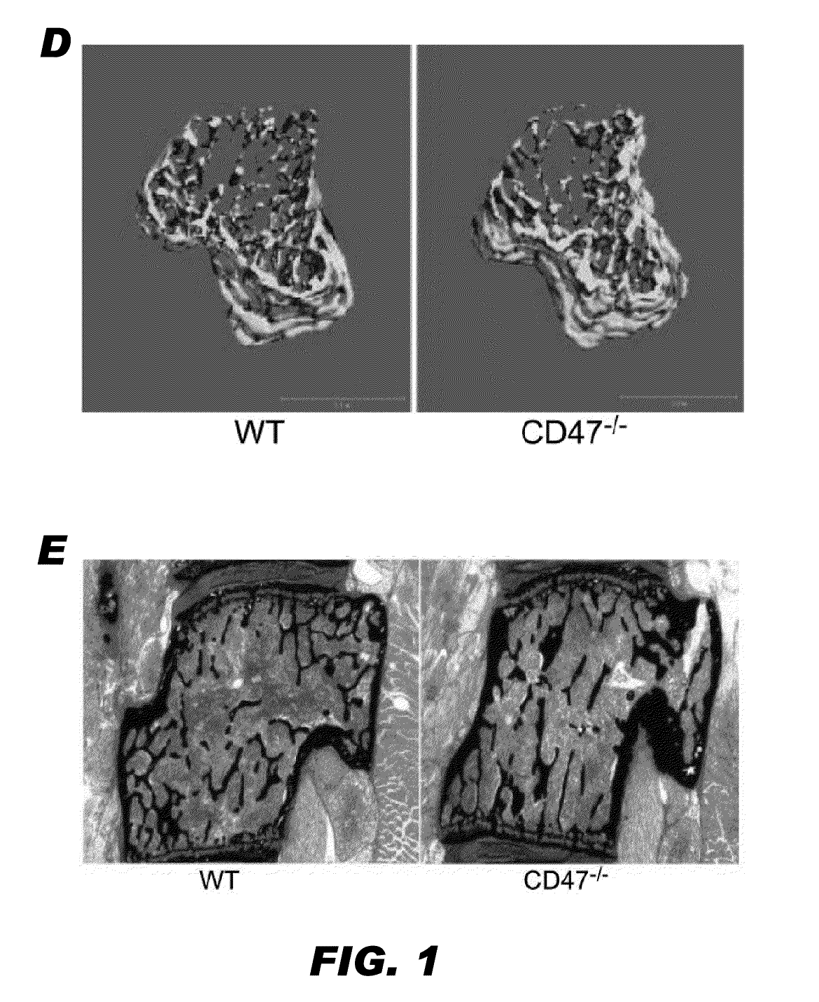

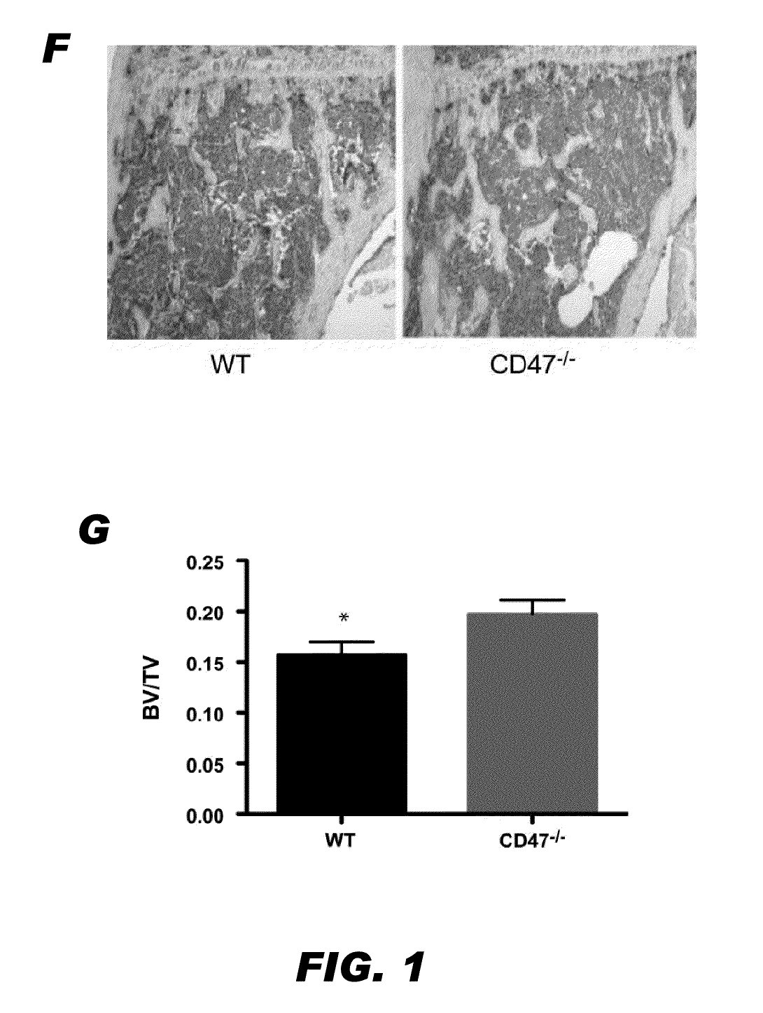

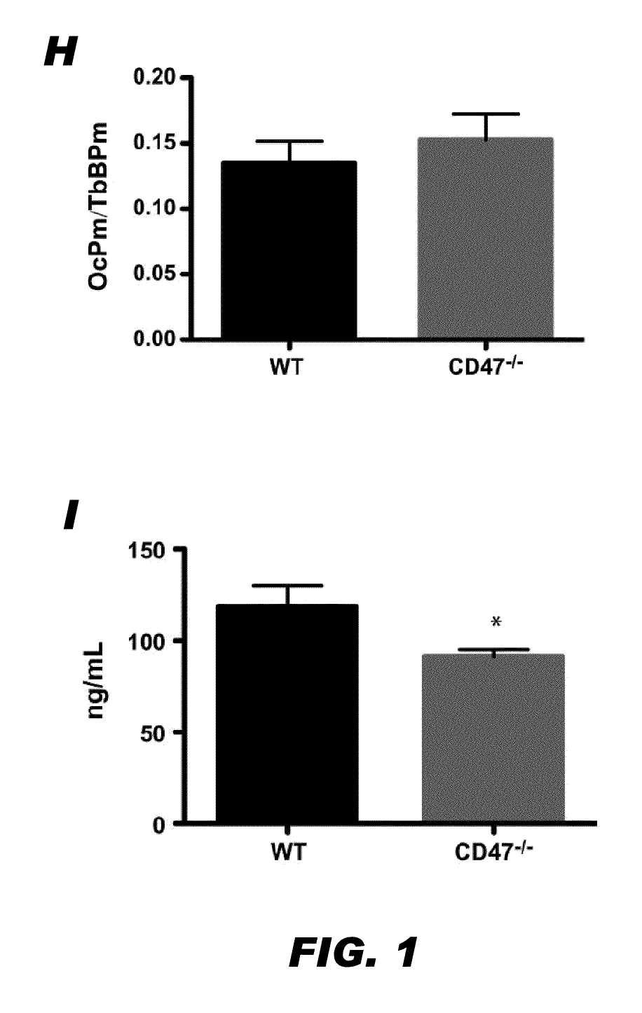

FIG. 1 shows that CD47−/− mice have Increased bone volume due to dysfunctional osteoclasts. A, B, C, D. WT and CD7−/− tibias were subjected to μCT analysis of bone parameters (WT n=5, CD47−/− n=5). A. Trabecular bone volume/total volume (BV/TV) by μCT (p=O.O4). B. Trabecular number by μCT (p=0.22), C. Trabecular thickness by μCT (p=0.04). D. Representative images of 3D μCT reconstruction of WT and CD47−/− tibias are shown. E. Lumbar vertebral bodies were stained with VonKossa. Black areas represent calcified bone. F. Representative histologicTRAP stained tibial sections are shown. G. BV/TV in tibias by histomorphometry (p=0.02). H. OC Perimeter/Trabecular Bone Perimeter (p=0.23). I. Collagen breakdown products (CTX) were measured in the serum of starved WT and CD47−/− mice (p=0.03).

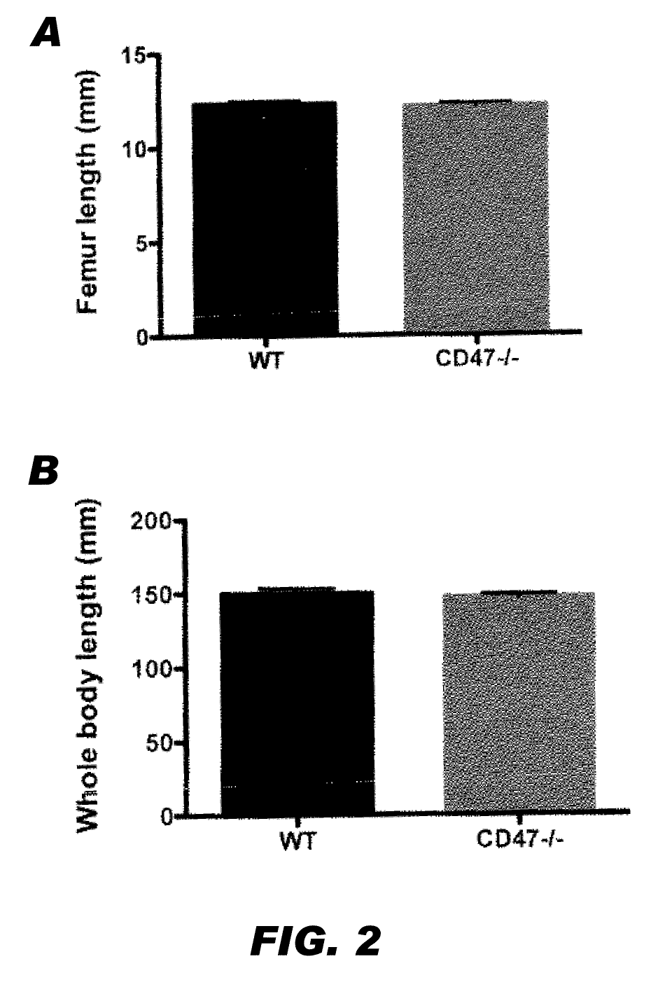

FIG. 2 shows that longitudinal growth is intact in CD47−/− mice. A. Isolated WT (n=8) and CD47−/− (n=8) femur length was measured by calipers. p=0.52. B. Whole body length of WT (n=4) and CD47−/− (n=4) mice was measured by calipers. p=0.55

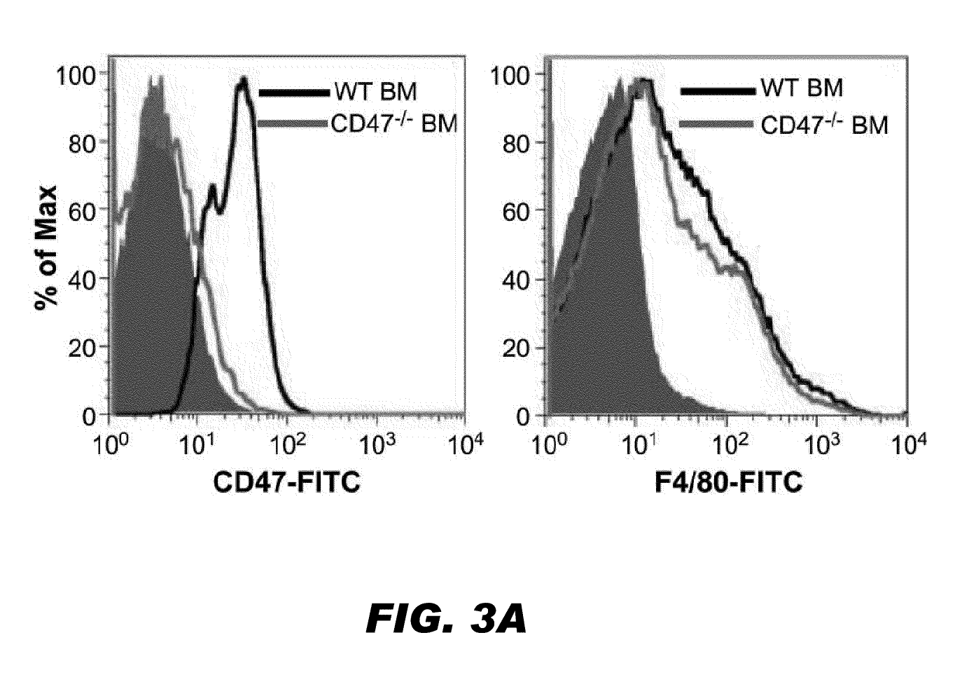

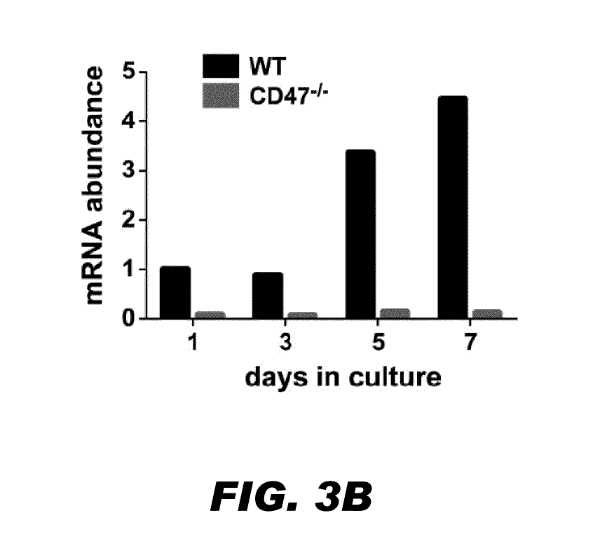

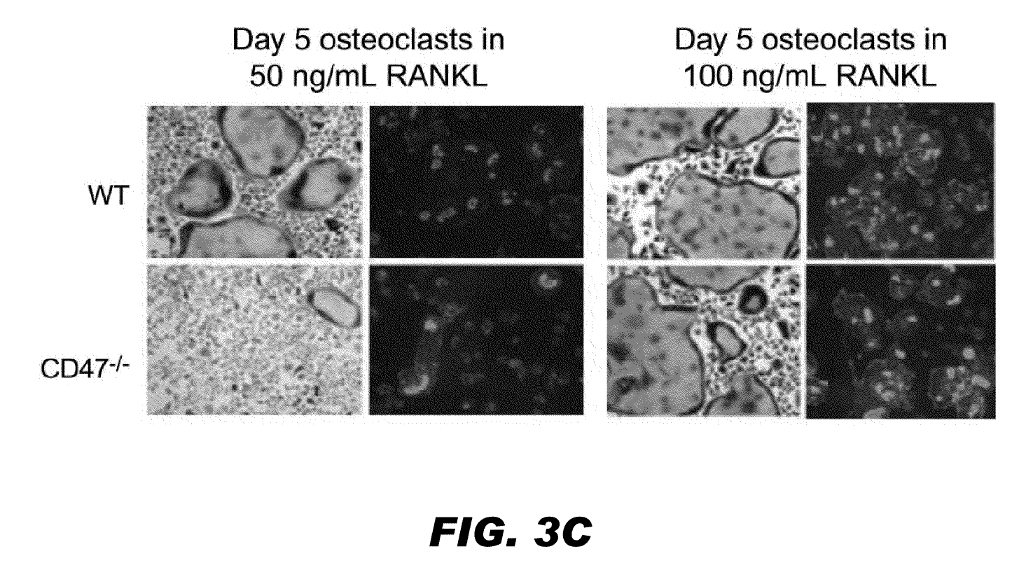

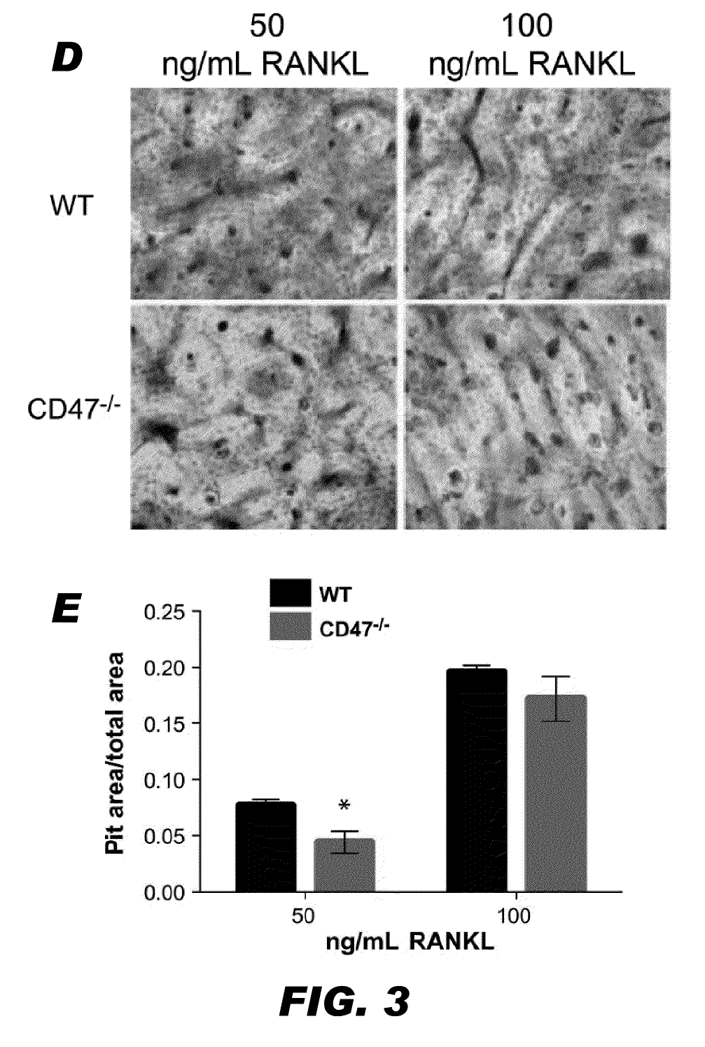

FIG. 3 shows that CD47−/− osteoclast defect can be rescued by high doses of RANKL in vitro. A. FACS analysis on WT and CD47−/− total bone marrow. WT and CD47−/− total bone marrow was stained for CD47 and F4/80. B. QPCR graph showing induction of CD47 transcript over the 7-day course of OC differentiation. C. Representative images of day 5 OCs cultured on bone and stained for TRAP (left panel) and cultured on bovine bone and stained for actin rings (right panel) in the presence of 50 ng/ml of M-CSF and 50 ng/ml or 100 ng/ml of RANKL are shown. D. Representative images of day 5 OCs cultured on bone and stained for resorption lacunae in the presence of 50 ng/ml of M-CSF and either 50 ng/ml of RANKL (left panel) and 100 ng/ml of RANKL (right panel) are shown. E. The pit (resorption lacunae) area was measured by histomorphometry. 50 ng/ml RANKL (p=0.04), 100 ng/ml RANKL (p=0.18).

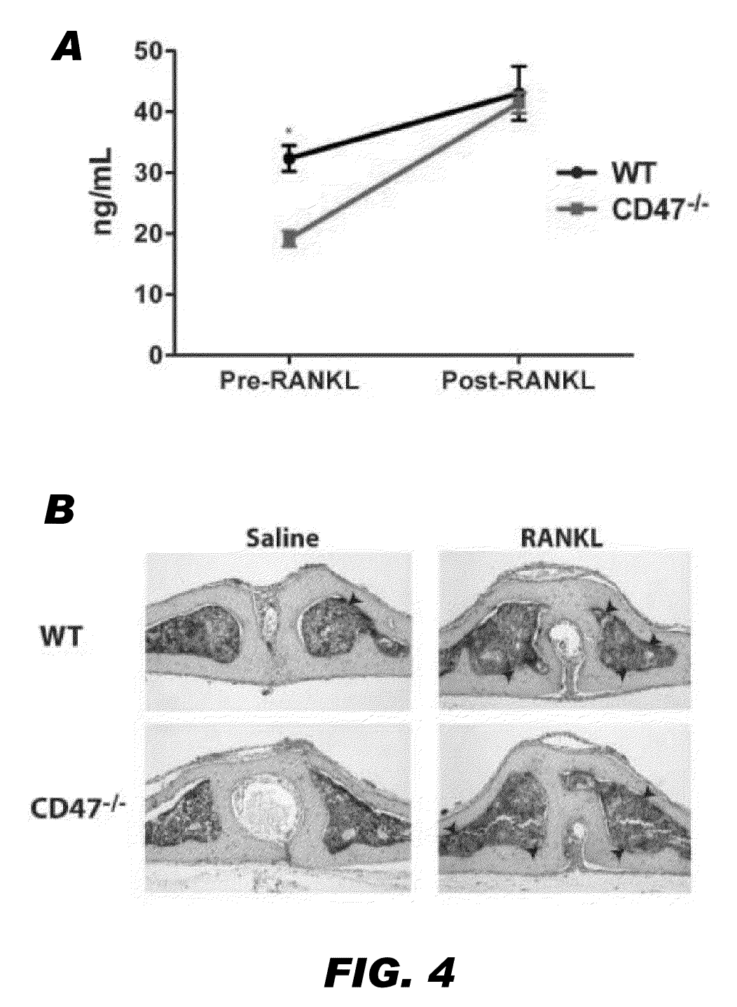

FIG. 4 shows that the osteoclast dysfunction in CD47−/− mice is rescued by in vivo RANKL injections. A. 100 μg of RANKL was delivered subperiostally onto the midline calvaria of WT (n=5) and CD47−/− (n=5) mice. Serum CTX was measured before and after RANKL injections. WT vs. CD47−/− pre-RANKL p<0.01, WT vs. CD47−/− post-RANKL p=0.74, WT pre-RANKL vs. post-RANKL p=0.03, CD47−/− pre-RANKL vs. post-RANKL p<0.01. B. Representative images of TRAP stained calvarial sections. Arrows point to recruited OCs.

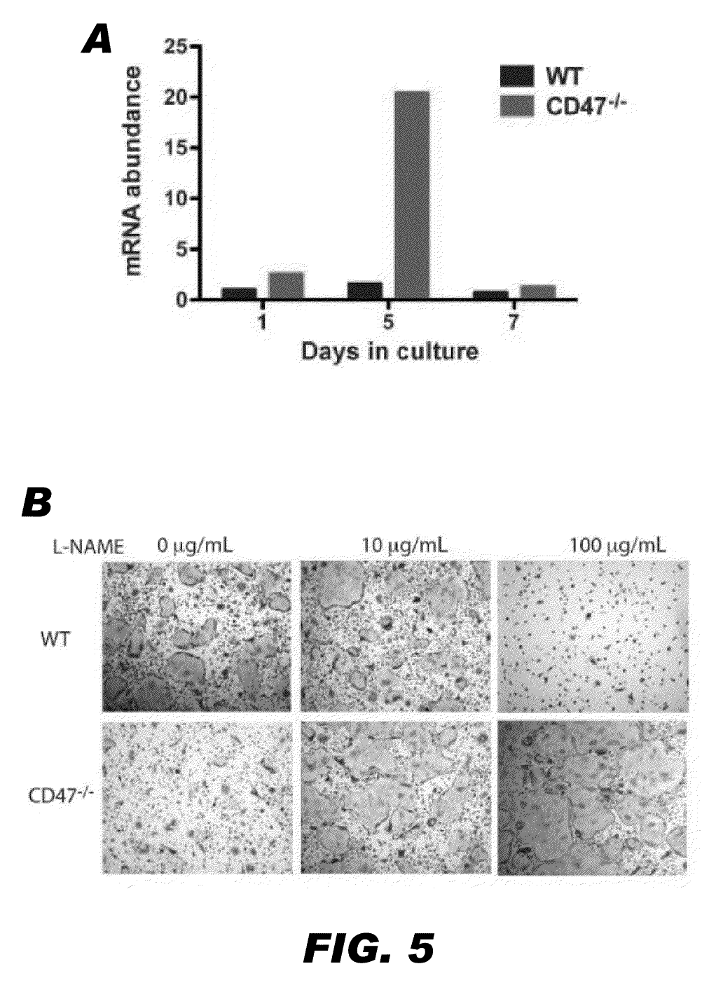

FIG. 5 shows that NOS inhibition restores OC differentiation in CD47−/− cells. A. WT and CD47−/− macrophages were differentiated into OC in the presence of 50 ng/ml M-CSF and 50 ng/ml of RANKL. RNA was isolated at days 1, 5 and 7 and cDNA was made. qPCR analysis was carried out with specific primers to iNOS. B. WT and CD47−/− macrophages were differentiated into OCs in the presence of M-CSF, RANKL and L-NAME, a pan inhibitor of NOS for 5 days.

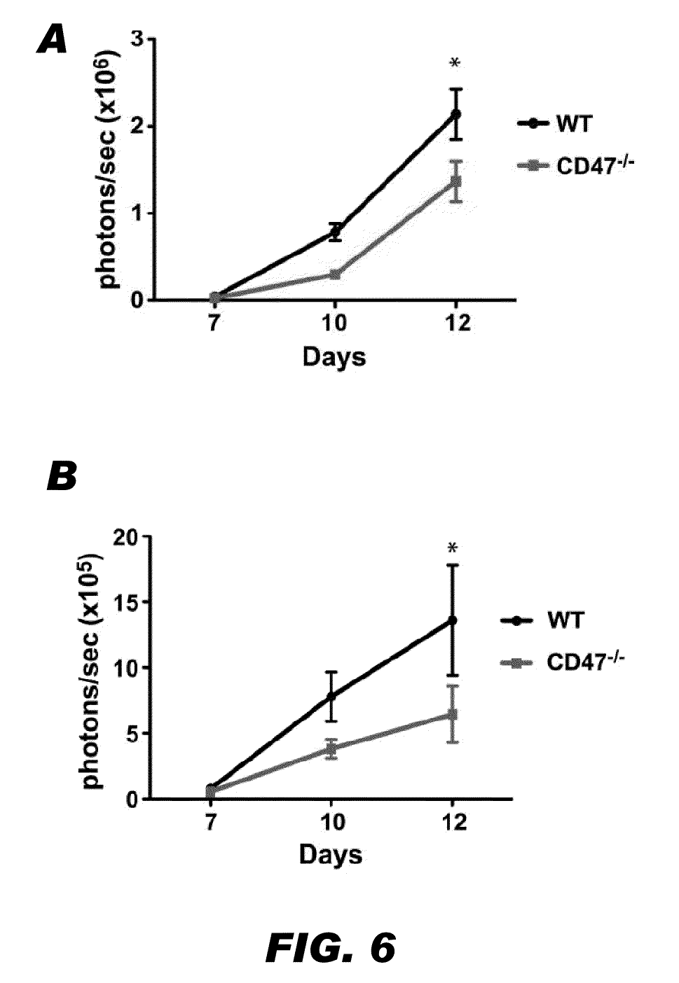

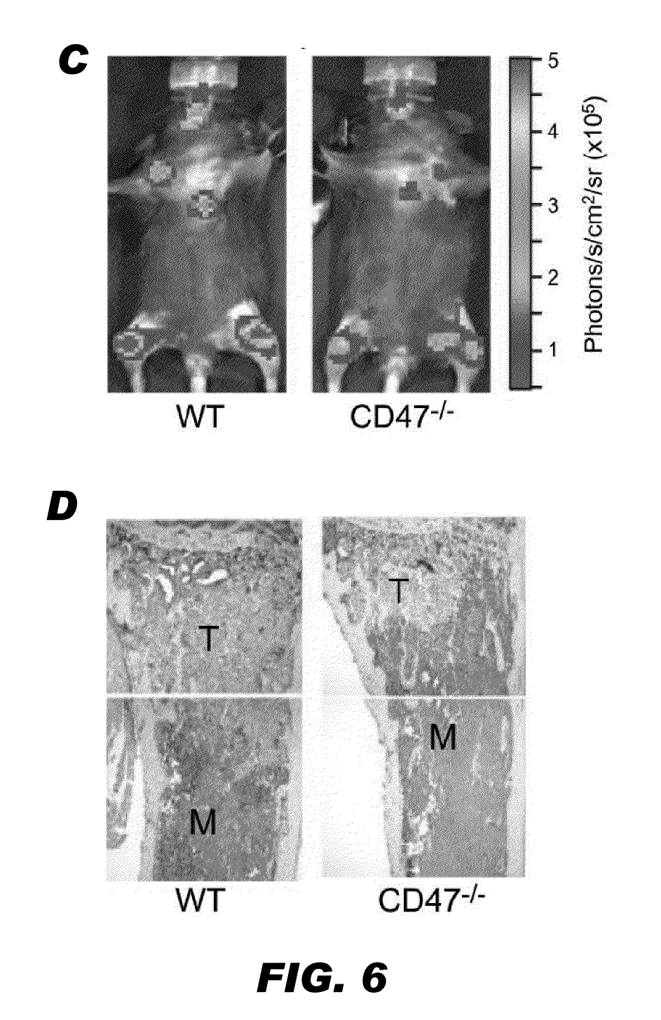

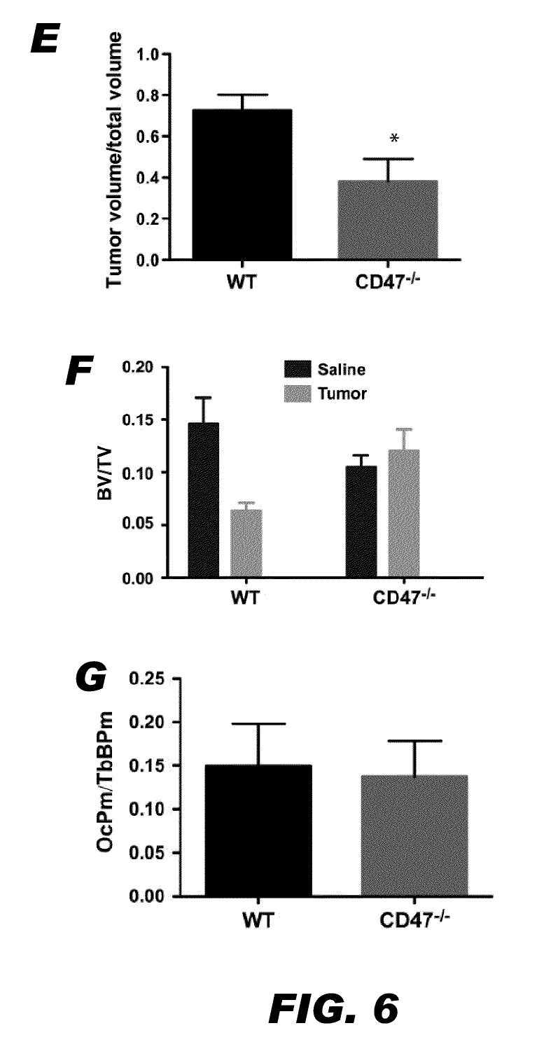

FIG. 6 shows that CD47−/− mice have decreased tumor burden and bone loss in an intracardiac metastasis model. A, B. B16-FL cells were injected into the left ventricular chamber of WT (n=3) and CD47−/− (n=3) mice. Tumor burden in the (A) femur/tibia (p=0.003) and (B) the mandible (p=0.05) as measured by bioluminescence imaging 7, 10 and 12 days post tumor cell injection. C. Representative bioluminescence images from day 12 are shown. D. Representative images of tibial histologic bone sections from day 12 are shown (M=Marrow, T=Tumor). E. Tumor volume/total volume in the tibias of WT (n=3) and CD47−/− (n=3) mice at day 12 were measured by histomorphometric analysis (p=0.02). F. Trabecular bone volume in the tibias of WT (n=3) and CD47−/− (n=3) mice at day 12 was measured by histomorphometric analysis. WT Saline vs. Tumor p<0.01, CD47−/− Saline vs. Tumor p=0.51. Three independent experiments showed similar results. G. Osteoclast perimeter on day 12.

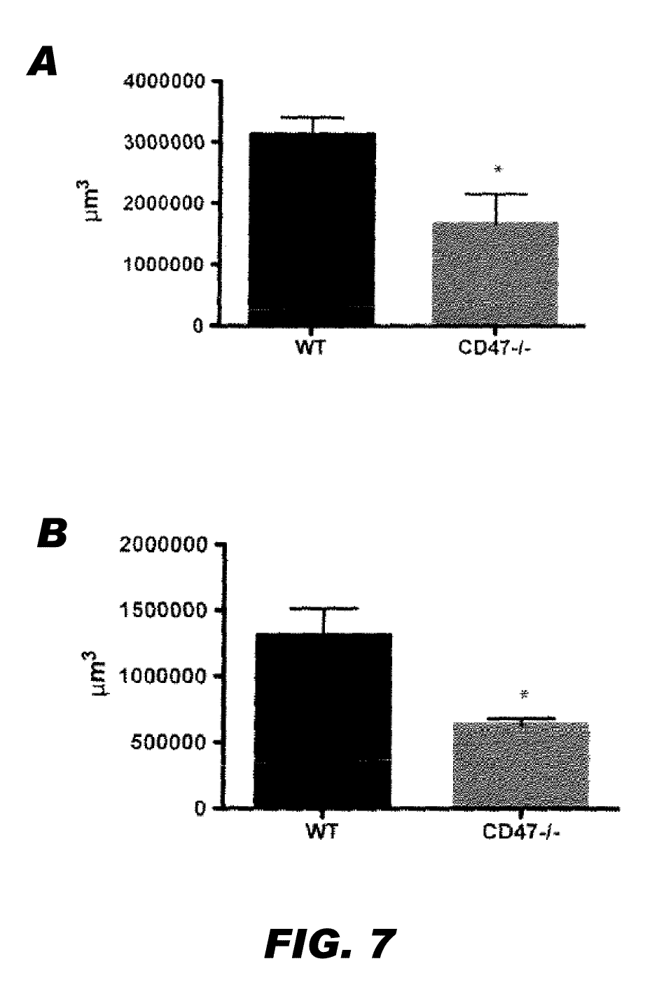

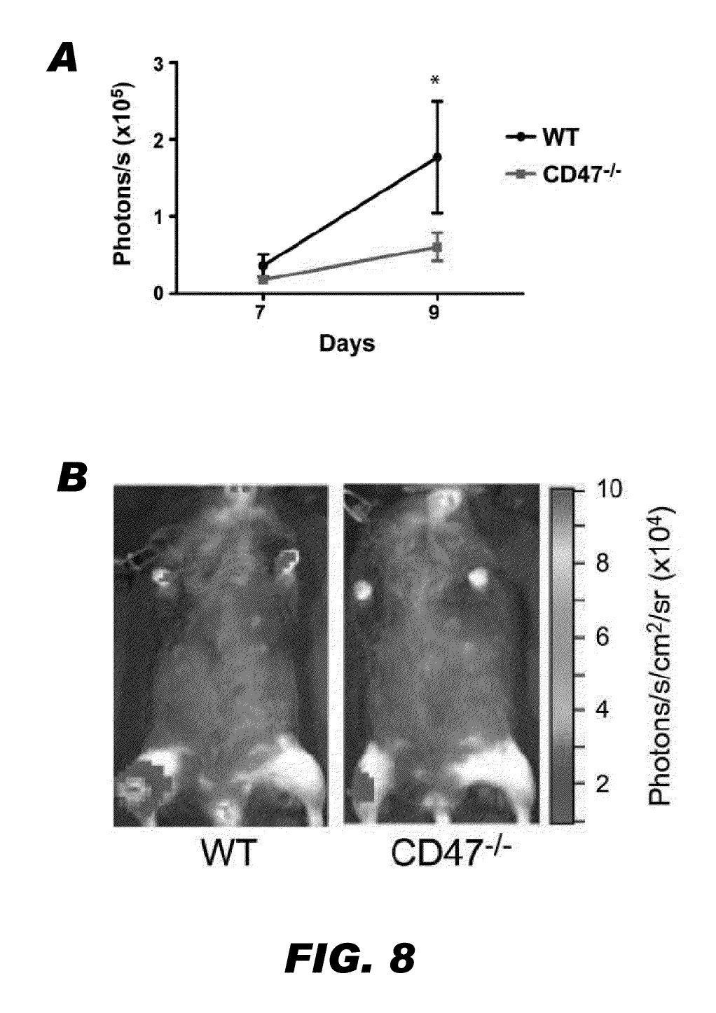

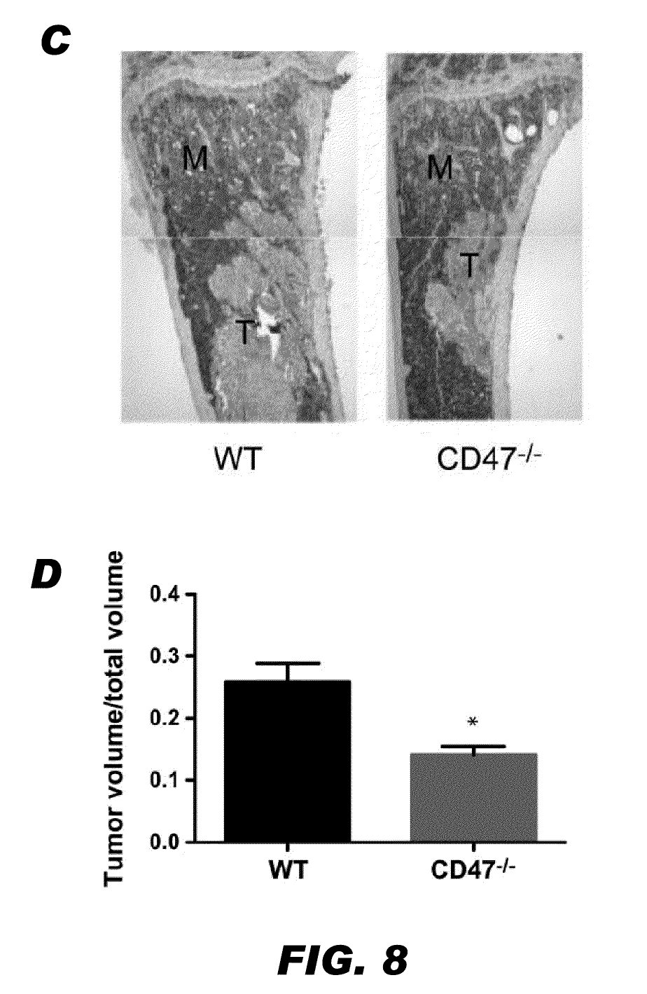

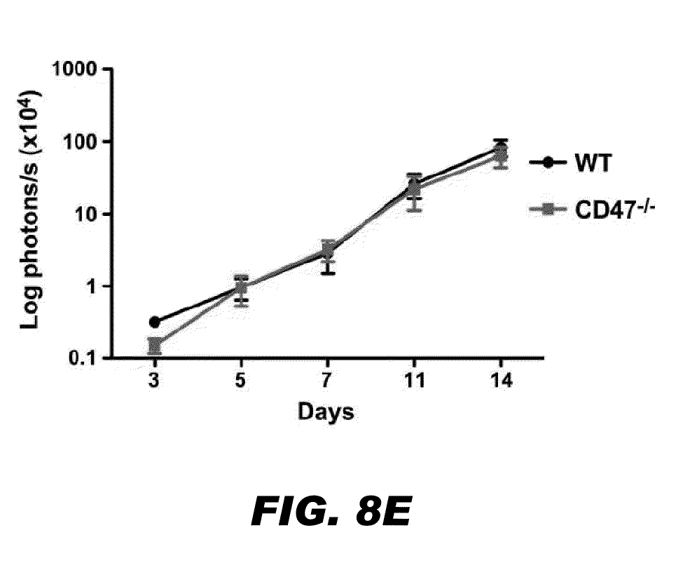

FIG. 7 shows that absolute tumor volume in bone is decreased in CD47−/− mice. A. Tumor volume from FIG. 6E is shown as absolute tumor volume (intra-arterial model). p=0.04. B. Tumor volume from FIG. 8D is shown as absolute tumor volume (intra-tibial model). P=0.04.

FIG. 8 shows that CD47−/− mice have decreased tumor burden and bone loss in an intratibial metastasis model but not in a subcutaneous model. A. B16-FL cells were injected directly into the tibia of WT (n=6) and CD47−/− (n=5) mice. Tumor burden in the tibiae was measured by bioluminescence imaging at days 7 and 9 after B16-FL injection (p=0.05). B. Representative bioluminescence images from day 9 are shown. C. Representative images of tibial histologic bone sections at day 9 are shown (M=Marrow, T=Tumor). D. Tumor volume/total volume in the tibias of WT (n=6) and CD47−/− (n=5) mice at day 9 were measured by histomorphometric analysis (p=0.03). Three independent experiments showed similar results. E. B16-FL cells were injected s.c into the hind flank of mice and tumor burden was measured by bioluminescence imaging at days 5, 7, 10 and 14 after B16-FL injection.

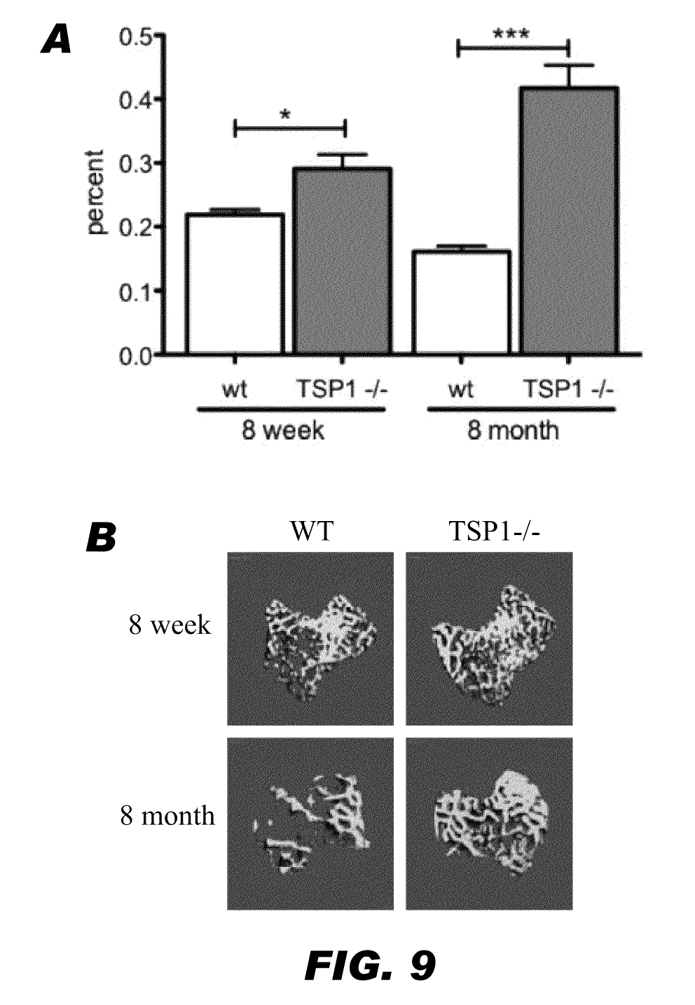

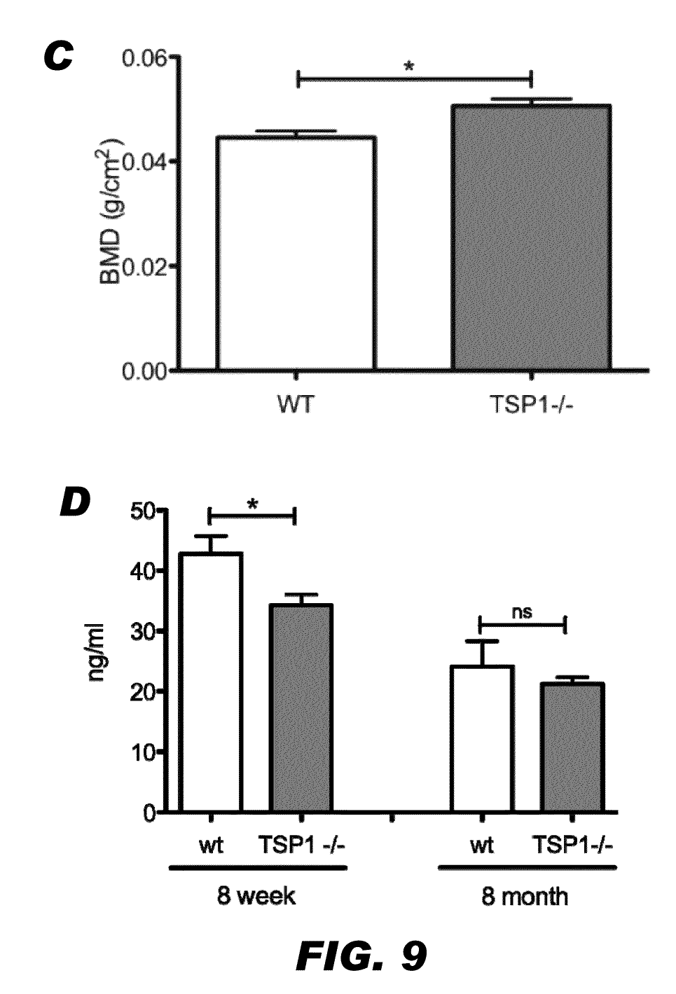

FIG. 9 shows that TSP1−/− mice show increased trabecular bone volume and decreased osteoclast function and bone mineral density compared to wild type controls. A. WT and TSP1−/− 8 week old femurs and 8 month old tibiae were subjected to microCT analysis. Trabecular BV/TV by microCT: 8 week (p=0.005), 8 month (p<0.0001). (B) Representative images of three-dimensional microCT reconstruction. (C) Bone mineral density measurements using DXA analysis in 9 week old WT and TSP1−/− mice (p=0.001). (D) Collagen breakdown products (CTX) were measured in the serum of starved WT and TSP1−/− mice; 8 week (p=0.02), 8 month (not significant).

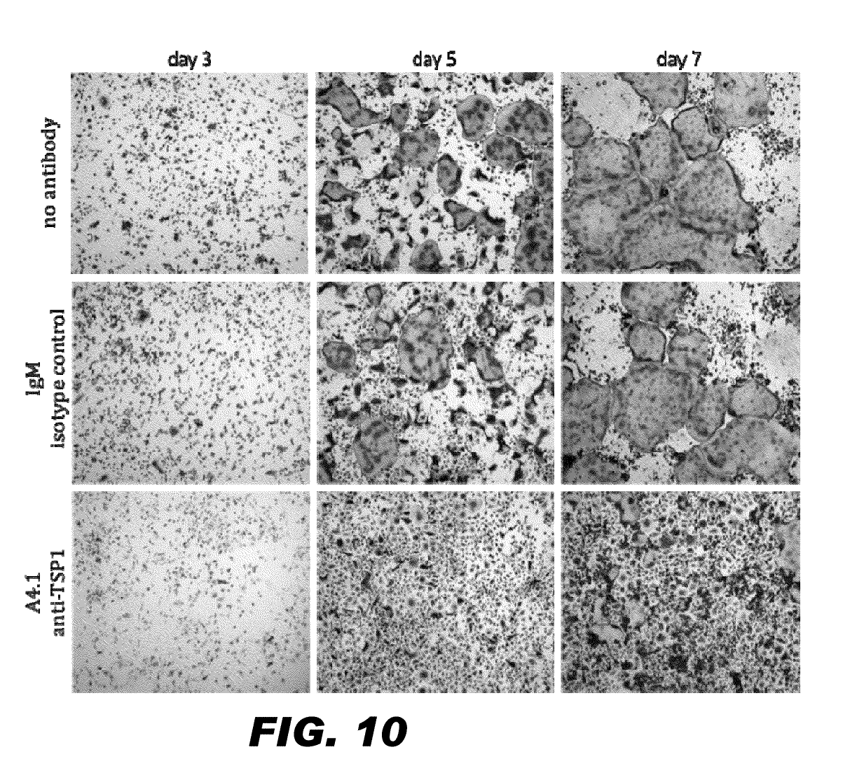

FIG. 10 shows that TSP1-deficient macrophages fail to form mature OC. Representative images of WT bone marrow macrophages cultured in the presence of 50 ng/ml M-CSF and 50 ng/ml RANK-L and treated with no antibody (top panels), IgM isotype control (middle panels), or A4.1 anti-TSP1 antibody (bottom panels). Cells TRAP stained (OC marker) at days 3, 5, and 7. TSP1-deficient macrophages fail to form mature OC.

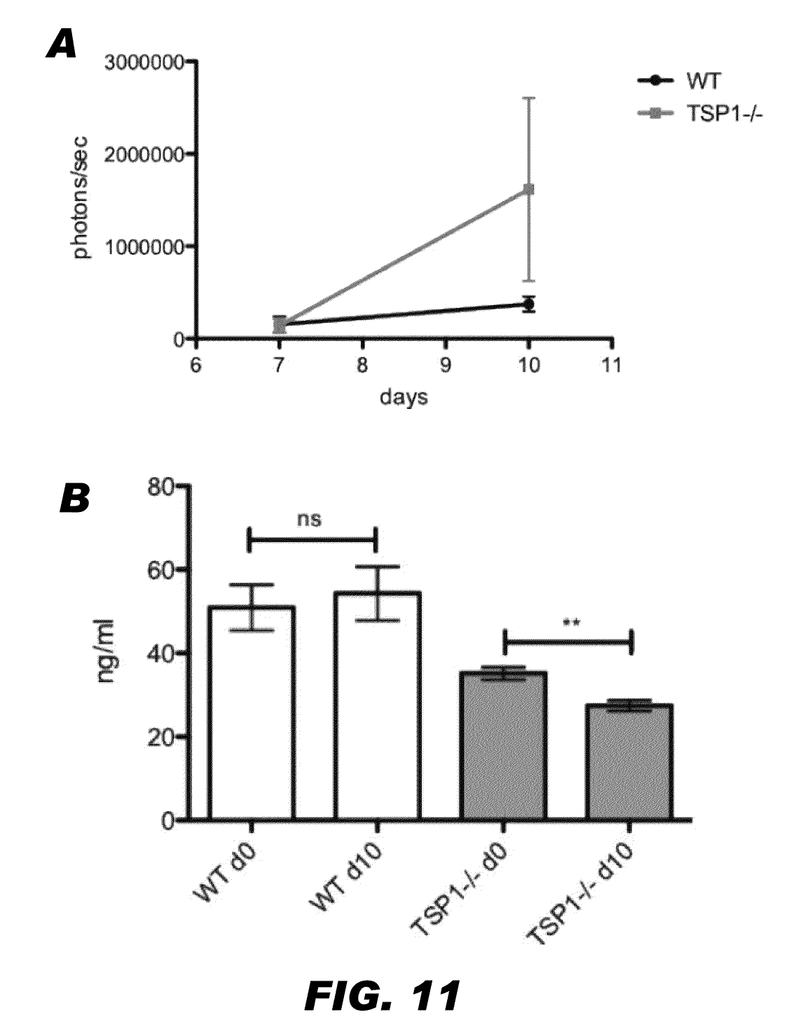

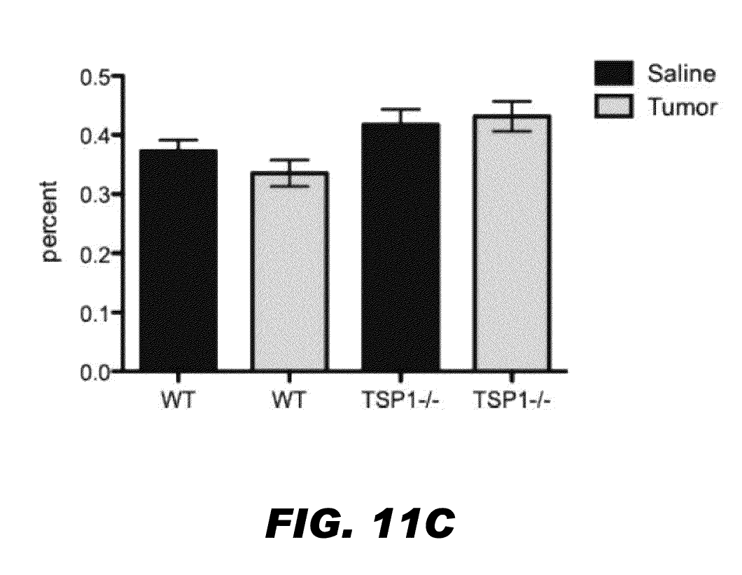

FIG. 11 shows that despite increased tumor burden, TSP1−/− mice show decreased osteoclast function and do not show decreased trabecular bone volume in an intrabial metastasis model. (A) B16-FL cells were injected directly into the tibia of WT and TSP1−/− mice. Tumor burden in the tibiae was measured by bioluminescence imaging at days 7 and d10 after B16-FL injection. (B) Serum CTX was measured in the serum of starved WT and TSP1−/− prior to (d0) and day 10 after (d10) B16-FL injection. Wild type (not significant); TSP1−/− (p=0.0016). (C) WT and TSP1−/− tibiae were subjected to microCT analysis at day 9 after B16-FL injection (black bars) or saline (gray bars).

### DETAILED DESCRIPTION OF THE INVENTION

### I. Indications

It has been discovered, as illustrated in the examples, that CD47, a receptor for Thrombospondin-1 and for signal inhibitory receptor proteins (transmembrane phosphatases) or SIRPs, plays a role in the development of functional osteoclasts, the cells that normally function to degrade bone. In particular, it has been discovered that by blocking the action of CD47 and/or TSP-1 and/or SIRPs, the functionality of osteoclasts can be reduced, leading to less bone destruction and hence preservation of bone mass. Exemplary bone loss associated disorders that may be treated by the method of the invention include, but are not limited to, osteoporosis, rickets, osteomalacia, McCune-Albright syndrome, and Paget's disease, as well as bone density loss promoted by the treatment of HIV/AIDs, autoimmune disease, epilepsy, juvenile rheumatoid arthritis, and the like.

In another aspect of this invention, it has been discovered that CD47 also plays a role in tumor growth in bone. In particular, it has been discovered that by blocking the action of CD47 and/or TSP-1 and or SIRPs, the tumor burden in bone can be reduced as well as bone degradation, leading to increased survival and decreased bone pain. Several tumor cell types metastasize to bone, and lead to bone degradation via activation of bone-resorbing osteoclasts (OC). There are many types of cancer cells that are known to metastasize. Examples of general categories of cancers include carcinomas, malignant tumors derived from epithelial cells represented by the most common cancers, including the common forms of breast, prostate, lung and colon cancer; sarcomas, malignant tumors derived from connective tissue, or mesenchymal cells; lymphomas and leukemias, malignancies derived from hematopoietic (blood-forming) cells, germ cell tumors, tumors derived from totipotent cells; blastic tumors or blastomas, tumors (usually malignant) which resembles an immature or embryonic tissue most common in children. In particular, exemplary tumor cell types that are known to metastasize to bone may include breast and prostate carcinomas and melanomas.

Typically, the method of the invention may be utilized for any mammalian subject. Such mammalian subjects include, but are not limited to, human subjects or subjects and companion animals. Exemplary companion animals may include domesticated mammals (e.g., dogs, cats, horses), mammals with significant commercial value (e.g., dairy cows, beef cattle, sporting animals), mammals with significant scientific value (e.g., captive or free specimens of endangered species), or mammals which otherwise have value.

### II. Methods for Inhibiting CD47

The present invention includes methods for inhibiting bone loss, bone metastasis and osteoclast differentiation by blocking the activity of CD47. In one embodiment, blocking the activity of CD47 means disrupting a signal transmitted intracellularly or extracellularly via CD47. In another embodiment, blocking the activity of CD47 means disrupting the signaling pathway that CD47 is involved in. Methods of measuring disruption of CD47 signaling are known in the art. Inhibiting the signaling function of CD47 directly may inhibit bone loss and bone metastasis. Alternatively, inhibiting the signaling function of other components of the signaling pathway that interact with CD47 may inhibit CD47 function. CD47 has been shown to interact directly with thrombospondin 1(TSP1). Other thrombospondins may include thrombospondin 2, 3, 4, and 5. TSP1 binds to CD47 via a peptide sequence in the TSP carboxyterminal domain—RFYWMWK—that is conserved among the products of all five TSP genes. CD47 has also been shown to interact directly with members of the SIRPα family of SIRPs. Other SIRPs that CD47 may interact with may include members of the SIRPβ family, such as SIRPβ2.

Non-limiting examples of suitable inhibiting agents include natural compounds, synthetic compounds, small organic compounds, peptides, peptide nucleic acids, peptidomimetics, antibodies, antisense oligonucleotides, aptamer oligonucleotides, morpholinos, or double-stranded RNA interference molecules. Furthermore, the inhibiting agent may be an individual compound or it may be a member of a combinatorial chemical library. A combinatorial chemical library is generally a collection of diverse chemical compounds generated by either chemical synthesis or biological synthesis by combining a number of chemical “building blocks.” For example, a linear combinatorial chemical library, such as a polypeptide library, may be formed by combining a set of amino acids in every possible way for a given length (i.e., the number of amino acids in a polypeptide agent). Millions of chemical compounds may be synthesized through such combinatorial mixing of chemical building blocks. Numerous combinatorial libraries are commercially available (e.g., ComGenex, Princeton, N.J.; Asinex, Moscow, Russia: Tripos, Inc., St. Louis, Mo.; ChemStar, Ltd, Moscow, Russia; 3D Pharmaceuticals, Exton, Pa.; Martek Biosciences, Columbia, Md.).

Examples of CD47 antagonists may be found in the patents WO/2008/060785, 20080131431, 20070113297, and 20060135749, each of which is hereby incorporated by reference in its entirety.

Peptides. The CD47 inhibitor may be a peptide. A CD47-binding peptide from the C-terminal module of TSP1, p7N3 (FIRWMYEGKK) was shown to be effective against CD47 activity. Stated another way, the peptide blocked (or disrupted) CD47 signaling. Another peptide from TSP1 that was found to bind CD47 has the following sequence IGWKDFTAYR. Derivatives of this peptide have the potential to act as inhibitors of TSP1-CD47 binding or SIRP-CD47 binding. This and related peptides were described in the patent WO/2008/060785, which is hereby incorporated by reference in its entirety. In addition, a recombinant form of the CBD (rCBD) has been expressed and shown to compete with binding of TSP1 with CD47, thus inhibiting the function of CD47 in vitro. A SIRPα-binding peptide, CERVIGTGWVRC that structurally mimics an epitope on CD47 and binds to SIRP has been shown to inhibit CD47 activity in vitro.

A skilled practitioner will recognize that peptides may be substantially similar to the peptides described above in that an amino acid residue may be substituted with another amino acid residue having a similar side chain without affecting the function of the peptide. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Thus, the peptide inhibitor may have one or more conservative amino acid substitutions.

The degree of sequence identity between two amino acid sequences may be determined using the BLASTp algorithm of Karlin and Altschul (Proc. Natl. Acad. Sci. USA 87:2264-2268, 1993). The percentage of sequence identity is determined by comparing two optimally aligned sequences over a comparison window, wherein the portion of the amino acid sequence in the comparison window may comprise additions or deletions (i.e., gaps) as compared to the reference sequence (which does not comprise additions or deletions) for optimal alignment of the two sequences. The percentage is calculated by determining the number of positions at which an identical amino acid occurs in both sequences to yield the number of matched positions, dividing the number of matched positions by the total number of positions in the window of comparison and multiplying the result by 100 to yield the percentage of sequence identity.

The fragment or fragments of digested thrombospondin 1 may be purified and isolated using techniques that are well known in the art. Alternatively, the peptide inhibitor of CD47 may be recombinantly produced from DNA encoding sequences using molecular biology techniques well know to those with skill in the art. The recombinant peptide may be produced in bacterial cell, eukaryotic cells, or mammalian cells. The peptide inhibitor may also be synthesized in vitro using solid phase synthesis techniques that are well known in the art. Guidance for any of the above-mentioned techniques may be found in reference texts such as Current Protocols in Molecular Biology (Ausubel et al., John Wiley & Sons, New York, 2003) or Molecular Cloning: A Laboratory Manual (Sambrook & Russell, Cold Spring Harbor Press, Cold Spring Harbor, N.Y., 3rd edition, 2001).

Antibodies. In other embodiments, the CD47 signaling inhibitor may be an antibody or a fragment thereof. In some embodiments, the antibody that inhibits CD47 signaling may be a single chain antibody. The single chain antibody may be a single chain Fv (scFv) fragment in which the variable regions of the light and heavy chains are joined by a flexible linker moiety. The single chain Fv antibody may be generated using methods disclosed in U.S. Pat. No. 4,946,778 or using phage display library techniques (Huse et al., 1989, Science 246:1275-1281; McCafferty et al., 1990, Nature 348:552-554) (each of these is incorporated in its entirety by reference). In other embodiments, the antibody that inhibits CD47 may be an antibody fragment. Suitable antibody fragments include Fab fragments, Fab′ fragments, Fd fragments (i.e., heavy chain variable domain), and Fv fragments. These antibody fragments may be generated by enzymatic cleavage, via recombinant libraries, expression libraries, phage display techniques, or other means known to those of skill in the art (for additional guidance, see e.g., Coico, R. (ed), Current Protocols in Immunology, 2007, John Wiley & Sons, Inc., New York). In yet other embodiments, the antibody that that inhibits CD47 may be a camelid antibody, which is a small antibody molecule that lacks light chains (Hamers-Casterman et al., 1993, Nature 363(6428):446-448). In further embodiments, the antibody that inhibits CD47 may be a chimeric antibody or antibody fragment. Alternatively, the antibody that inhibits CD47 may be a humanized antibody or antibody fragment. Those of skill in the art are familiar with techniques to generate chimeric or humanized antibodies.

In some embodiments, the antibody that inhibits CD47 may bind directly to CD47. Inhibitory antibodies that block CD47 function has been described (Parkos, et al., 1996. J. Cell Biol. 132:437; Gao et al., 1996. J. Cell. Biol., 135: 533-544; Frazier et al., 1999. J. Biol. Chem., 274: 8554-8560; Manna et al., 2003. J. Immunol., 170: 3544-3553; Green et al., 1999. J. Cell Biol., 146: 673-682), each of which is hereby incorporated by reference in its entirety. These antibodies include C5D5, 1F7, 2D3, and B6H12 and the mAbs 400, 410, 420, 430, 440, 450, 460. 470. 480 (Han et al, J. Biol. Chem. 275:37984-37992, 2000), which is hereby incorporated by reference in its entirety. In other embodiments, the antibody that inhibits CD47 may bind thrombospondins. Inhibitory antibodies that block TSP1 function has been described (Wang, et al., 1996. Surgery, 120(2):449-54;), each of which is hereby incorporated by reference in its entirety. In yet other embodiments, the antibody that inhibits CD47 may bind SIRPs.

Inhibition of expression. CD47 activity may be inhibited by inhibiting expression of CD47 or the expression of interaction partners TSPs and SIRPs. Expression may be disrupted using an RNA interference (RNAi) agent that inhibits expression of a target mRNA or transcript. The RNAi agent may lead to cleavage of the target transcript. Alternatively, the RNAi agent may prevent or disrupt translation of the target transcript into a protein.

In some embodiments, the RNAi agent may be a short interfering RNA (siRNA). In general, a siRNA comprises a double-stranded RNA molecule that ranges from about 15 to about 29 nucleotides in length. The siRNA may be about 16-18, 17-19, 21-23, 24-27, or 27-29 nucleotides in length. In a preferred embodiment, the siRNA may be about 21 nucleotides in length. The siRNA may optionally further comprise one or two single-stranded overhangs, e.g., a 3′ overhang on one or both ends. The siRNA may be formed from two RNA molecules that hybridize together or, alternatively, may be generated from a short hairpin RNA (shRNA) (see below). In some embodiments, the two strands of the siRNA may be completely complementary, such that no mismatches or bulges exist in the duplex formed between the two sequences. In other embodiments, the two strands of the siRNA may be substantially complementary, such that one or more mismatches and/or bulges may exist in the duplex formed between the two sequences. In certain embodiments, one or both of the 5′ ends of the siRNA may have a phosphate group, while in other embodiments one or both of the 5′ ends lack a phosphate group. In other embodiments, one or both of the 3′ ends of the siRNA may have a hydroxyl group, while in other embodiments one or both of the 5′ ends lack a hydroxyl group.

One strand of the siRNA, which is referred to as the “antisense strand” or “guide strand,” includes a portion that hybridizes with the target transcript. In preferred embodiments, the antisense strand of the siRNA may be completely complementary with a region of the target transcript, i.e., it hybridizes to the target transcript without a single mismatch or bulge over a target region between about 15 and about 29 nucleotides in length, preferably at least 16 nucleotides in length, and more preferably about 18-20 nucleotides in length. In other embodiments, the antisense strand may be substantially complementary to the target region, i.e., one or more mismatches and/or bulges may exist in the duplex formed by the antisense strand and the target transcript. Typically, siRNAs are targeted to exonic sequences of the target transcript. Those of skill in the art are familiar with programs, algorithms, and/or commercial services that design siRNAs for target transcripts. An exemplary example is the Rosetta siRNA Design Algorithm (Rosetta Inpharmatics, North Seattle, Wash.) and MISSION® siRNA (Sigma-Aldrich, St. Louis, Mo.). The siRNA may be enzymatically synthesized in vitro using methods well known to those of skill in the art. Alternatively, the siRNA may be chemically synthesized using oligonucleotide synthesis techniques that are well known in the art.

In other embodiments, the RNAi agent may be a short hairpin RNA (shRNA). In general, an shRNA is an RNA molecule comprising at least two complementary portions that are hybridized or are capable of hybridizing to form a double-stranded structure sufficiently long to mediate RNA interference (as described above), and at least one single-stranded portion that form a loop connecting the regions of the shRNA that form the duplex. The structure may also be called a stem-loop structure, with the stem being the duplex portion. In some embodiments, the duplex portion of the structure may be completely complementary, such that no mismatches or bulges exist in the duplex region of the shRNA. In other embodiments, the duplex portion of the structure may be substantially complementary, such that one or more mismatches and/or bulges may exist in the duplex portion of the shRNA. The loop of the structure may be from about 1 to about 20 nucleotides in length, preferably from about 4 to about 10 about nucleotides in length, and more preferably from about 6 to about 9 nucleotides in length. The loop may be located at either the 5′ or 3′ end of the region that is complementary to the target transcript (i.e., the antisense portion of the shRNA).

The shRNA may further comprise an overhang on the 5′ or 3′ end. The optional overhang may be from about 1 to about 20 nucleotides in length, and more preferably from about 2 to about 15 nucleotides in length. In some embodiments, the overhang may comprise one or more U residues, e.g., between about 1 and about 5 U residues. In some embodiments, the 5′ end of the shRNA may have a phosphate group, while in other embodiments it may not. In other embodiments, the 3′ end of the shRNA may have a hydroxyl group, while it other embodiments it may not. In general, shRNAs are processed into siRNAs by the conserved cellular RNAi machinery. Thus, shRNAs are precursors of siRNAs and are similarly capable of inhibiting expression of a target transcript that is complementary of a portion of the shRNA (i.e., the antisense portion of the shRNA). Those of skill in the art are familiar with the available resources (as detailed above) for the design and synthesis of shRNAs. An exemplary example is MISSION® shRNAs (Sigma-Aldrich).

In still other embodiments, the RNAi agent may be an RNAi expression vector. Typically, an RNAi expression vector may be used for intracellular (in vivo) synthesis of RNAi agents, such as siRNAs or shRNAs. In one embodiment, two separate, complementary siRNA strands may be transcribed using a single vector containing two promoters, each of which directs transcription of a single siRNA strand (i.e., each promoter is operably linked to a template for the siRNA so that transcription may occur). The two promoters may be in the same orientation, in which case each is operably linked to a template for one of the complementary siRNA strands. Alternatively, the two promoters may be in opposite orientations, flanking a single template so that transcription for the promoters results in synthesis of two complementary siRNA strands. In another embodiment, the RNAi expression vector may contain a promoter that drives transcription of a single RNA molecule comprising two complementary regions, such that the transcript forms an shRNA.

Those of skill in the art will appreciate that it is preferable for siRNA and shRNA agents to be produced in vivo via the transcription of more than one transcription unit. Generally speaking, the promoters utilized to direct in vivo expression of the one or more siRNA or shRNA transcription units may be promoters for RNA polymerase III (Pol III). Certain Pol III promoters, such as U6 or H1 promoters, do not require cis-acting regulatory elements within the transcribed region, and thus, are preferred in certain embodiments. In other embodiments, promoters for Pol II may be used to drive expression of the one or more siRNA or shRNA transcription units. In some embodiments, tissue-specific, cell-specific, or inducible Pol II promoters may be used.

A construct that provides a template for the synthesis of siRNA or shRNA may be produced using standard recombinant DNA methods and inserted into any of a wide variety of different vectors suitable for expression in eukaryotic cells. Guidance may be found in Current Protocols in Molecular Biology (Ausubel et al., John Wiley & Sons, New York, 2003) or Molecular Cloning: A Laboratory Manual (Sambrook & Russell, Cold Spring Harbor Press, Cold Spring Harbor, N.Y., 3^{rd} edition, 2001). Those of skill in the art also appreciate that vectors may comprise additional regulatory sequences (e.g., termination sequence, translational control sequence, etc.), as well selectable marker sequences. DNA plasmids are known in the art, including those based on pBR322, PUC, and so forth. Since many expression vectors already contain a suitable promoter or promoters, it may be only necessary to insert the nucleic acid sequence that encodes the RNAi agent of interest at an appropriate location with respect to the promoter(s). Viral vectors may also be used to provide intracellular expression of RNAi agents. Suitable viral vectors include retroviral vectors, lentiviral vectors, adenoviral vectors, adeno-associated virus vectors, herpes virus vectors, and so forth. In preferred embodiment, the RNAi expression vector is an shRNA lentiviral-based vector or lentiviral particle, such as that provided in MISSION® TRC shRNA products (Sigma-Aldrich).

The RNAi agents or RNAi expression vectors may be introduced into the cell using methods well known to those of skill in the art. Guidance may be found in Ausubel et al., supra or Sambrook & Russell, supra, for example. In some embodiments, the RNAi expression vector, e.g., a viral vector, may be stably integrated into the genome of the cell, such that sialidase expression is disrupted over subsequent cell generations.

In other embodiments, homologous recombination techniques may be used to disrupt sialidase expression at the level of the genomic DNA. Accordingly, these techniques may be used to delete a gene, delete a portion of a gene, or introduce point mutations in the gene, such that no functional product may be made. In one embodiment, the gene may be targeted by homologous recombination using the techniques of Capecchi (Cell 22:4779-488, 1980) and Smithies (Proc Natl Acad Sci USA 91:3612-3615, 1994). In other embodiments, the gene may be targeted using a Cre-loxP site-specific recombination system, an Flp-FRT site-specific recombination system, or variants thereof. Such recombination systems are commercially available, and additional guidance may be found in Ausubel et al., supra. In still another embodiment, the gene may be targeted using zinc finger nuclease (ZFN)-mediated gene targeting (Sangamo Biosciences, Richmond, Calif.). Briefly, ZFNs are synthetic proteins comprising an engineered zinc finger DNA-binding domain fused to the cleavage domain of the FokI restriction endonuclease. ZFNs may be used to induce double-stranded breaks in specific DNA sequences and thereby promote site-specific homologous recombination and targeted manipulation of genomic sequences. ZFNS may be engineered to target any DNA sequence of interest.

In other embodiments, antisense oligonucleotides, or similar antisense technology such as morpholinos. Antisense molecules interact with complementary strands of nucleic acids, modifying expression of genes. Antisense oligonucleotides may be RNA or DNA. Morpholinos are synthetic molecules that are the product of a redesign of natural nucleic acid structure. Structurally, the difference between Morpholinos and DNA is that while Morpholinos have standard nucleic acid bases, those bases are bound to morpholine rings instead of deoxyribose rings and linked through phosphorodiamidate groups instead of phosphates. Replacement of anionic phosphates with the uncharged phosphorodiamidate groups eliminates ionization in the usual physiological pH range, so Morpholinos in organisms or cells are uncharged molecules. The entire backbone of a Morpholino is made from these modified subunits. Morpholinos are most commonly used as single-stranded oligos, though heteroduplexes of a Morpholino strand and a complementary DNA strand may be used in combination with cationic cytosolic delivery reagents. Morpholinos that inhibit CD47 expression, or the expression of TSP have been described (Isenberg, et al., 2007. Circulation Research; 100:712; Isenberg, et al., 2007. Arteriosclerosis, Thrombosis, and Vascular Biology. 27:2582; Isenberg et al., 2009. J. Biol. Chem., Vol. 284, 1116-1125), each of which is hereby incorporated by reference in its entirety.

Decoy receptors. A decoy receptor, or sink receptor, is a receptor that binds a ligand, inhibiting it from binding to its normal receptor. Decoy receptors that inhibit CD47 function have been described. Decoy receptors may consist of the extracellular IgV domain of CD47 affixed to any other protein sequence that promotes its correct expression, export, folding and stability in the extracellular milieu. Also the IgV domain of SIRP-alpha which binds to CD47 may be used as a decoy to block CD47 binding to TSP1 or other ligands (such as cell associated SIRPs).

### III. Combination Therapy

The method of the present invention may also be administered as a combination therapy with any other drug or agent known in the art to have utility for inhibiting bone loss, bone metastasis and osteoclast differentiation. In one embodiment, the agent having utility for inhibiting bone loss, bone metastasis and osteoclast differentiation is an antimetabolite including folate antagonists (e.g. methotrexate), pyrimidine antagonists (e.g. cytarabine, floxuridine, fludarabine, fluorouracil, and gemcitabine), purine antagonists (e.g. cladribine, mercaptopurine, thioguanine), and adenosine deaminase inhibitors (e.g. pentostatin). In an alternative embodiment, the agent having utility for inhibiting bone loss, bone metastasis and osteoclast differentiation is an alkylating agent such as chlorambucil, cyclophosphamide, busulfan, ifosfamide, melphalan, and thiotepa. In yet another embodiment, the agent having utility for inhibiting bone loss, bone metastasis and osteoclast differentiation is an alkylator agent such as cisplatin, carboplatin, procarbazine, dacarbazine, and altretamine. In still another embodiment, the antineoplastic agent is an anti-tumor antibiotic such as bleomycin, dactinomycin, and mitomycin. In yet a further embodiment, the agent having utility for inhibiting bone loss, bone metastasis and osteoclast differentiation is an immunological agent such as interferon. In another embodiment, the agent having utility for inhibiting bone loss, bone metastasis and osteoclast differentiation is a plant alkaloid including vinca alkaloids (e.g. vinblastine, vincristine and vinorelbine), epipodophyllotoxins (e.g. etoposide and teniposide), taxanes (e.g. docetaxel and paclitaxel), and camptothecins (e.g. topotecan and irinotecan).

In an additional embodiment, the agent having utility for inhibiting bone loss, bone metastasis and osteoclast differentiation is a bisphosphonate including etidronate (Didronel), clodronate (e.g. Bonefos, Loron), tiludronate (e.g. Skelid), pamidronate (e.g. APD, Aredia), neridronate, olpadronate, alendronate (e.g. Fosamax), ibandronate (e.g. Bonviva), risedronate (e.g. Actonel), and zoledronate (e.g. Zometa, Aclasta). Of course those skilled in the art will appreciate that the particular agents having utility for inhibiting bone loss, bone metastasis and osteoclast differentiation to be administered with the compound(s) of the invention will vary considerably depending on the type of bone loss, bone metastasis and osteoclast differentiation disorder being treated and its stage of progression.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### CD47−/− Mice have Increased Bone Volume Due to Dysfunctional Osteoclasts

CD47 interacts with and activates β3 integrins. β3−/− mice display osteopetrosis due to osteoclast (OC) dysfunction. Therefore, it was hypothesized that CD47−/− mice might have an OC defect. To test this hypothesis, μCT analysis on tibias from 8-week old WT and CD47−/− littermates was performed. We observed an increase in trabecular bone volume/total volume in CD47−/− mice compared to WT mice (FIG. 1A, D). There was no change in trabecular number but the trabecular thickness was increased in CD47−/− tibias (FIG. 1B, C, D). VonKossa staining of the lumbar vertebral bodies, a marker for mineralized matrix confirmed an increase in bone volume and trabecular thickness in CD47−/− mice (FIG. 1E). The increase in trabecular bone volume (BV/TV) was also confirmed by histomorphometry on histological sections (FIG. 1F, G). There was no difference in longitudinal growth of CD47−/− mice as measured by femur length and whole body length (FIG. 2). Interestingly, it was also observed that OC perimeter/total trabecular bone perimeter was not changed in CD47−/− mice in vivo (FIG. 1H). To confirm that this increase in BV/TV was due to dysfunctional OCs, serum CTX was measured, the C-terminal telopeptide of collagen type I that is cleaved upon bone resorption by OCs. A decrease in CTX activity was observed (FIG. 1I), which along with an increase in trabecular bone volume suggests that the OC activity was decreased in CD47−/− mice in vivo (FIG. 1I).

### Example 2

### CD47−/− Osteoclast Defect can be Rescued by High Doses of RANKL In Vitro

To investigate if the OC defect is cell-autonomous in CD474−/− mice, it was first determined if macrophages or OC precursors were decreased in CD47−/− bone marrow. WT and CD47−/− whole bone marrow were stained with CD47-FITC as control and F4/80 as a macrophage marker and carried out flow cytometry. No significant difference in F4/80+cells in CD47−/− bone marrow compared to WT control was observed (FIG. 3A). To test the differentiation of macrophages to OCs, we cultured whole bone marrow in M-CSF alone for 3 days to enrich for macrophages. An equal number of macrophages were then cultured in the presence of M-CSF and RANKL to promote differentiation into OCs for 7 days. At a dose of 50 ng/ml of RANKL, we observed induction of CD47 transcript levels over the course of OC differentiation in WT but not in CD47−/− cells (FIG. 3B). We observed that WT macrophages produced large, multi-nucleated, TRAP-positive OCs by day 5, but only a few OCs formed from CD47−/− macrophages. When WT and CD47−/− macrophages were plated onto bovine bone slices, there was a decrease in the number of multi-nucleated CD47−/− OCs with multiple actin rings visualized by phalloidin staining (FIG. 3C). However, when the dose of RANKL was increased two-fold to 100 ng/ml, the OC differentiation defect was largely rescued in CD47−/− cells. This was reflected in equivalent numbers of OCs with multiple actin rings formed on bovine bone from macrophages of both genotypes (FIG. 3C). To determine the functional capacity of CD47−/− OCs to resorb bone, we stained the bones with wheat-germ agglutinin to measure the areas of resorption lacunae after 5 days on bovine bone. There was a significant decrease in bone resorption by CD47−/− OCs in 50 ng/ml RANKL; however, the resorption capacity of CD47−/− OCs was similar to WT at a high dose of RANKL (FIG. 3E. F).

### Example 3

### The Osteoclast Dysfunction in CD47−/− Mice was Rescued by In Vivo RANKL Injections

To determine if RANKL could rescue CD47−/−OC function in vivo as was observed in vitro, we injected 100 mg of RANKL subperiostally into the midline calvaria of 8-week old WT and CD47−/− mice. We measured serum CTX pre and post-RANKL injections in these mice. Pre-RANKL injection, CD47−/− mice had lower serum CTX as we have shown before (FIG. 4A). However, after RANKL administration, OC activation was not significantly different between WT and CD47−/− mice as measured by serum CTX (FIG. 4A). Furthermore, direct visualization of OC recruitment by TRAP staining on histological sections of calvarial bone in WT and CD47−/− mice confirmed the CTX results (FIG. 4B). We conclude that a high dose of RANKL rescued the cell-autonomous OC defect in CD47−/− mice.

### Example 4

### NOS Inhibition Restores OC Differentiation in CD47−/− cells

A number of tissues from CD47−/− mice have increased nitric oxide (NO) levels. We differentiated WT and CD47−/− macrophages into OCs in the presence of M-CSF and RANKL. We observed that iNOS expression levels were higher in CD47−/−OC cultures compared to WT controls (FIG. 5A). It has been previously shown that increased levels of NO lead to a block in OC differentiation. We hypothesized that this increase in NO levels is responsible for the inhibition of OC differentiation in CD47−/− cells. We differentiated WT and CD47−/− macrophages into OCs in the presence of L-NAME, a pan NOS inhibitor. In CD47−/− cells, we observed a dose-dependent rescue of OC differentiation with L-NAME administration. We observed a bi-phasic effect of L-NAME on WT cells, where at a low dose of L-NAME, there was modest enhancement of OC formation, and at a high dose of L-NAME, there was inhibition of OC differentiation (FIG. 4B). The OC inhibitory dose of L-NAME on WT cells was enhanced in CD47−/− cells. Taken together, these data support that the increased levels of iNOS in CD47−/− cells negatively affect OC formation.

### Example 5

### CD47−/− Mice have Decreased Tumor Burden and Bone Loss in an Intra-Cardiac Metastasis Model

The data above indicate that, at high doses of RANKL, CD47 was not necessary for OC differentiation and function. RANKL is produced from T-cells and osteoblasts during inflammatory conditions such as arthritis and bone metastasis. RANKL is not expressed by B 16-FL cells. We thus examined bone metastasis and osteolysis in CD47−/− mice to determine if this local increase in RANKL is able to rescue the CD47−/− OCs in this pathophysiological context. We evaluated bone metastasis in WT and CD47−/− mice using murine melanoma B16-F10 cells engineered to express firefly luciferase (B 16-FL). We measured bone tumor burden by real-time bioluminescence (BLI) on days 7, 10 and 12 after intra-cardiac B 16-FL injection, a route of administration that allows for bone metastasis rather than lung infiltration of injected cells. There was a significant decrease in tumor burden in the femoral/tibial bones and the mandible of CD47−/− mice compared to WT mice (FIG. 6A, B, C) as measured by BLI and confirmed by histomorphometric measurement of tumor volume in histological sections at day 12 post B16-FL inoculation (FIG. 6D, F and FIG. 7A). Tumor cells are known to secrete factors that activate OCs to degrade bone and induce osteolysis, which is a significant outcome of bone metastases. Trabecular bone volume was measured in WT and CD47−/− mice injected with B 16-FL or saline. While there was significant bone loss in B 16-FL in WT mice, there was no bone loss in CD47−/− bones injected with B 16-FL, consistent with the OC dysfunction in CD47−/− mice (FIG. 6D, F). We did not observe a difference in OC perimeter/trabecular bone perimeter in tumor-bearing CD47−/− bones (FIG. 6G). Thus, we conclude that CD47−/− mice had decreased bone tumor burden and tumor-associated bone destruction, and the local increase in RANKL levels in vivo was not sufficient to rescue the OCT defect in CD47−/− mice.

### Example 6

### CD47−/− Mice have Decreased Tumor Burden and Bone Loss in an Intra-Tibial Metastasis Model but not in a Subcutaneous Model

We have previously shown that platelets are critical for the homing of tumor cells to bone. CD47−/− mice have been shown to have a mild decrease in platelet numbers. To further confirm that this decrease in tumor burden in CD47−/− mice after intra-cardiac injections of B 16-FL cells was not due to compromised homing of tumor cells to bone, we turned to a more direct model of late-stage bone metastasis that eliminates initial tumor-homing steps of the metastatic process. We injected equal numbers of B 16-FL cells directly into the tibiae of WT and CD47−/− littermates and performed BLI on days 7 and 9 after B 16-FL inoculation. Bioluminescence imaging showed decreased tumor burden over time in the CD47−/− mice compared to WT mice (FIG. 8A, B). Histomorphometric measurement of tumor volume in histological sections at day 9 post B 16-FL inoculation confirmed this decrease (FIG. 8C, D and FIG. 7B).

To confirm that the decrease in bone tumor burden in CD47−/− mice was specific to CD47 function in the bone microenvironment and not a result of intervention by the immune system or tumor-associated angiogenesis in the CD47−/− mice, we measured local tumor burden in subcutaneously (s.c) injected B 16-FL cells. We observed no change in s.c tumor burden as measured by BLIO on days 5, 7, 10 and 14 at which point the experiment was terminated due to necrotic tumors (FIG. 8E). We conclude that the decrease in tumor burden in CD47−/− mice was specific to the bone compartment and not due to compromised immunity or platelet interactions.

### Materials and Methods for Examples 1-6

### Cells

The B16-F10 C57BL/6 murine melanoma cell line was purchased from American Type Culture Collection (ATCC; Manassas, Va.) and modified to express firefly luciferase (B16-FL).

### Animals

CD47−/− mice on a pure CS7BL/6 background were housed under pathogen-free conditions according to the guidelines of the Division of Comparative Medicine, Washington University School of Medicine. The animal ethics committee approved all experiments. For generation of CD47−/− mice, CD47+/− females were crossed to CD47+/− males and the females were allowed to have two litters.

### MicroCT (μCT)

Tibias and femurs were suspended in agarose and the right proximal tibial and femoral metaphyses were scanned by m CT (μ CT-40; Scanco Medical, Bassersdorf, Switzerland) as described previously. For image acquisition, the tibias were placed in a 17-mm holder and scanned. The image consisted of 50 slices. The trabecular region was selected using contours inside the cortical shell on each two-dimensional image: The growth plate was used as a marker to determine a consistent location to start analysis. A 3D cubical voxel model of bone was built, and the following calculations were made: relative bone volume over total bone volume (BV/TV), trabecular number and thickness. A threshold of 300 (out of 1000) was used to differentiate trabecular bone from non-bone.

### Histology, Bone Histomorphometry and Longitudinal Growth Measurements

Mouse tibias were fixed in formalin and decalcified in 14% EDTA. Paraffin-embedded sections were stained with hematoxylin and eosin, and separately for TRAP. Trabecular bone volume and tumor area were measured according to a standard protocol using Bioquant Osteo (Bioquant Image Analysis Corporation, Nashville Tenn.). Bone sections were blinded prior to analysis. Methylmethacrylate (MMA) embedded lumbar vertebral bodies of WT and CD47−/− mice were stained for VonKossa on a counter stain of 0.5% Basic Fuchsin. Longitudinal growth was measured by use of calipers on whole body as well as isolated femurs.

### Serum CFX Assay

CTX was measured from WT or CD47−/− mouse fasting serum by using a CTX ELISA system (Nordic Bioscience Diagnostics, Herlev, Denmark).

### Flow Cytometry (FACS)

Whole BM was isolated from WT and CD47−/− mice and incubated in blocking media (2.4G2 hybridoma with 4 ml of MsIgG). then incubated with FITC-conjugated anti-mouse F4/80 or anti-mouse CD47 (miap301) with an anti-rat FITC secondary antibody on ice for 20 mins. The cells were then washed twice and analyzed on a FACScan Flow Cytometer (BD Biosciences, San Jose, Calif.).

### In Vitro Osteoclast (OC) Assays

Whole bone marrow was isolated from wild-type C57BL16 mice and plated in M-CSF containing CMG-14-12 cell culture supernatant (1:10 vol) in α-MEM media containing 10% FBS in petri dishes for three days to generate primary bone marrow macrophages (BMMs). BMMs were lifted and equal numbers were plated in 48-well dishes in OC media: α-MEM containing 10% FBS, CMG-14-12 supernatant (1:20 vol), and GST-RANKL (50 ng/mL or 100 ng/mL) and incubated at 37° C. for 5 days to generate OCs. TRAP staining was performed according to manufacturers instructions (Sigma-Aldrich, St. Louis, Mo.). In FIG. 4, 0, 3, 10, 30 and 100 μg/ml of L-NAME (N-Nitro-L-Arginine Methyl Ester) was added to cultures at the same time with M-CSF and RANKL and media was changed every day. OC cultures were fixed after 4 days in culture and stained for TRAP.

### Reverse Transcription and Quantitative PCR

Reverse transcription and qPCR methods was carried out as described previously. qPCR primers for CD47: Forward GGCGCAAAGCACCGAAGAAATGTT (SEQ ID NO:1), Reverse-CCATGGCATCGCGCTTATCCATTT (SEQ ID NO:2), iNOS: Forward-GGCAGCCTGTGAGACCTTTG (SEQ ID NO:3), Reverse-GCATTGGAAGTGAAGCGTTTC (SEQ ID NO:4) and GAPDH: Forward TCAACAGCAACTCCCACTCTTCCA, (SEQ ID NO:5) Reverse-ACCCTGTTGCTGTAGCCGTATTCA (SEQ ID NO:6).

### Actin Ring Formation and Bone Resorption Assays

Actin ring formation and bone resorption assays were performed as described. Briefly, the cells plated on bovine bone slices were fixed with 3% paraformaldehyde in PBS for 20 mm. F-actin was stained with fluorescein isothiocyanate-labeled phalloidin at 0.3 μg/ml in PBS. For staining of the resorption lacunae (pits), the cells were brushed off the bone with a toothbrush. The slices were incubated with 20 μg/ml peroxidase-conjugated wheat germ agglutinin for one hour. After washing in PBS, 0.52 mg/ml of 3,3′-diaminohcnzidine with 0.1% H₂O₂ was added onto the bone slices for 15 minutes. Pit area was determined from five ×4 fields by using Osteo software (Bioquant, Nashville. TN) blinded to genotype.

### In Vivo RANKL Injections

100 μg of RANKL in a volume of 40 μl was injected subperiostially in the midline calvaria in 8 week-old mice once a day for 5 consecutive days. On the sixth day, serum was collected, mice were sacrificed and the calvarial bone was isolated. TRAP staining was performed on fixed, decalcified and paraffin-embedded calvarial bone.

### Tumor and Bone Metastasis Models

For intra-cardiac injections, the operator was blinded to genotype. Mice were anesthetized and inoculated intra-cardially via the left ventricular chamber with 10⁵ B16-FL cells in 504 PBS as previously described. Bioluminescence imaging was performed on days 7, 10 and 12 post B 16-FL cell inoculation. Mice were sacrificed and underwent blinded necropsy on day 12 after tumor cell injection. Mice were discarded from the final analysis if the animal died before day 12 or if necropsy demonstrated a large mediastinal tumor indicative of injection of tumor cells into the chest cavity, not the left ventricle.

For intra-tibial injections, mice were anesthetized, and 1×10⁴ B16-FL cells in 50 μL PBS was injected into the right tibia. PBS (50 μL) was injected into the left tibia as an internal control. Animals were radiographed in 2 dimensions using an X-ray system to confirm intratibial placement of the needle (Faxitron Corp, Buffalo Grove, Ill.). Bioluminescence imaging was performed on days 7 and 9 post B16-FL cell inoculation. Mice were sacrificed and underwent necropsy on day 9-post B16-FL inoculation. Mice with intramuscular locations of tumors were discarded from the analysis. For subcutaneous (s.c) injections, mice were anesthetized and 5×10⁵ B16-FL cells in 100 μL PBS were injected subcutaneously on the dorsal surface of the mouse at two sites. Tumor growth was monitored over the 14-day period following B16-FL injections, and bioluminescence imaging was performed 5, 7, 10 and 14 days post B16-FL inoculation. The experiment was terminated due to the presence of large, necrotic tumors.

### In Vivo Bioluminescence Imaging

Mice were injected intraperitoneally with 150 mg/kg D-luciferin (Biosynthesis, Naperville, Ill.) in PBS 10 minutes prior to imaging. Imaging was performed using a charged coupled device (CCD) camera (IVIS 100; exposure time of 1 or 5 minutes, binning of 8, filed of view [FOV] of 15 cm, f/stop of 1, and no filter) in collaboration with the Molecular Imaging Center Reporter Core (Washington University, St Louis). Mice were anesthetized by isoflurane (2% vaporized in O₂), and C57BL/6 mice were shaved to minimize attenuation of light by pigmented hair. For analysis, total photon flux (photons per second) was measured from a fixed region of interest (ROI) in the tibia/femur, the mandible or the local s.c tumor using Living Image 2.50 and Igor Pro software (Wavemetrics, Portland, Oreg.).

### Example 7

### TSP1 is a Ligand for CD47 and TSP1 Disruption Increases Bone Mass and Decreases OC Function as Observed in CD47−/− Mice

Contrary to CD47 itself, CD47 interacting molecules (TSP1 and (33 integrins) enhance bone mass and can prevent bone loss associated with pathologic bone disease, but these molecules also cause off-target effects on neo-blood vessels that may worsen tumor growth. For instance, TSP1−/− mice have increased tumor growth in bone. CD47 disruption is NOT associated with enhanced tumor angiogenesis which is accompanied by increased tumor growth supporting CD47 as a molecular target for treatment of pathologic bone disease and tumor metastasis.

TSP1−/− mice have increased trabecular bone volume compared to wild type controls, a difference that increases with age. Eight-week old TSP1 −/− mice have decreased CTX, indicating reduced osteoclast function, than wild type controls, and show increased bone mineral density. Thus TSP1−/− mice are protected from age associated bone loss. TSP1 blockade with TSP1 blocking antibody, disrupts osteoclast formation (FIG. 10). These data demonstrate that TSP1 plays a role in OC biology.

However, TSP1−/− have INCREASED tumor growth in bone despite being protected from tumor associated bone loss. There is increased tumor growth in the bones of TSP1−/− mice after intratibial injection of B16 melanoma cells as measured by bioluminescence compared to WT mice (FIG. 11). In contrast, the TSP1−/− mice were protected from tumor associated bone loss compared to WT mice despite the increased tumor burden. These data demonstrate that TSP1−/− mice do have osteoclast defects which protect them from tumor induced bone loss, however, the tumors grow bigger and faster in the TSP1−/− mice. This increased tumor growth that has been observed by others is likely due to the enhanced tumor associated blood vessel formation.

What is claimed is:

1. A method of inhibiting bone loss, the method comprising blocking CD47 signaling.

2. The method of claim 1, wherein bone loss is inhibited in a subject in need of treatment for a tumor.

3. The method of claim 1, wherein bone loss is inhibited in a subject in need of treatment for a tumor that has metastasized to the bone of the subject.

4. The method of claim 3, wherein the subject is administered an agent that blocks CD47 activity.

5. The method of claim 4, wherein the agent is an antibody.

6. A method of inhibiting bone metastasis, the method comprising blocking CD47 signaling.

7. The method of claim 6, wherein bone loss is inhibited in a subject in need of treatment for a tumor that has metastasized to the bone of the subject.

8. The method of claim 7, wherein the subject is administered an agent that blocks CD47 activity.

9. The method of claim 8, wherein the agent is an antibody.

10. A method of inhibiting osteoclast differentiation, the method comprising blocking CD47 signaling.

11. The method of claim 10, wherein osteoclast differentiation is inhibited in a subject in need of treatment for a tumor that has metastasized to the bone of the subject.

12. The method of claim 11, wherein the subject is administered an agent that blocks CD47 activity.

13. The method of claim 12, wherein the agent is an antibody.
